(19) United States
(12) Patent Application Publication
(10) Pub. No.: US 2019/0038555 A1
(43) Pub. Date: Feb. 7, 2019

(54) BICONTINUOUS MICROEMULSIONS AND METHODS OF USE THEREOF

(71) Applicant: The Regents of the University of California, Oakland, CA (US)

(72) Inventors: Meng C. Lin, Dublin, CA (US); Tatyana F. Svitova, Emeryville, CA (US)

(21) Appl. No.: 16/068,535

(22) PCT Filed: Feb. 24, 2017
(86) PCT No.: PCT/US17/19425

§ 371 Date: Jul. 6, 2018

Related U.S. Application Data
(60) Provisional application No. 62/300,245, filed on Feb. 26, 2016.

(51) Int. Cl. A61K 9/107; A61K 9/00

(52) U.S. Cl. CPC A61K 9/1075; A61K 9/0048

(57) ABSTRACT

The present disclosure provides bicontinuous microemulsions, and methods of using the bicontinuous microemulsions in the treatment of dry eye.

### CROSS-REFERENCE

This application claims the benefit of U.S. Provisional Patent Application No. 62/300,245, filed Feb. 26, 2016, which application is incorporated herein by reference in its entirety.

### INTRODUCTION

The sensation of ocular discomfort referred to as “dry eye” can be caused by various factors. The principle causative factors are: a) inadequate tear production attributable to aging, medical procedures performed on the cornea, or other conditions; b) environmental irritants (e.g., dust, smoke, wind, sun, low humidity); and c) eye strain attributable to extended viewing of computer monitors, or to other workplace environment-related factors.

### SUMMARY

The present disclosure provides bicontinuous microemulsions, and methods of using the bicontinuous microemulsions in the treatment of dry eye.

The present disclosure provides a bicontinuous microemulsion comprising: a) an aqueous phase comprising: i) sodium chloride; ii) sea salt; iii) ascorbic acid; and iv) trehalose; and b) a lipid phase comprising one or more of: i) sphingomyelin; ii) phosphatidylcholine; iii) fish oil; iv) coconut oil; v) grapeseed oil; vi) beeswax; vii) ghee; and viii) lanolin, wherein the osmolarity of the microemulsion is from 270 mOsm to 320 mOsm, and wherein the microemulsion has a pH of from 7.2 to 7.4. In some cases, the ratio of aqueous phase to lipid phase is from about 10:1 to 1:10 (v/v). In some cases, the ratio of aqueous phase to lipid phase is from about 5:1 to 1:5 (v/v). In some cases, the ratio of aqueous phase to lipid phase is from about 3:1 to 1:3 (v/v). In some cases, the ratio of aqueous phase to lipid phase is from about 2.5:1 to 3:1 (v/v). In some cases, the lipid phase of the bicontinuous microemulsion comprises palm oil and fish oil. In some cases, the lipid phase of the bicontinuous microemulsion comprises palm oil, fish oil, and coconut oil. In some cases, the lipid phase of the bicontinuous microemulsion comprises grapeseed oil, coconut oil, beeswax, sphingomyelin, and phosphatidylcholine. In some cases, the lipid phase of the bicontinuous microemulsion comprises palm oil, beeswax, lecithin, and phosphatidylcholine. In some cases, the lipid phase of the bicontinuous microemulsion comprises ghee, lecithin, and phosphatidylcholine. In some cases, the bicontinuous microemulsion further comprises beeswax. In some cases, the bicontinuous microemulsion further comprises palm oil. In some cases, the bicontinuous microemulsion further comprises olive oil. In some cases, the bicontinuous microemulsion further comprises palm oil and olive oil. In some cases, the bicontinuous microemulsion further comprises ghee. In some cases, the bicontinuous emulsion is sterile. The present disclosure provides a container comprising a bicontinuous microemulsion as described above or elsewhere herein. In some cases, the container is an eyedropper. In some cases, the container is sterile; and the bicontinuous emulsion is sterile. The present disclosure provides a method of reducing evaporation of an aqueous tear film from the surface of an eye, the method comprising applying a bicontinuous microemulsion, as described above or elsewhere herein, to the surface of the eye, thereby reducing evaporation of the aqueous tear film from the surface of the eye. The present disclosure provides a method of treating dry eye in an eye of an individual, the method comprising applying a bicontinuous microemulsion, as described above or elsewhere herein, to the surface of the eye of the individual. In some cases, the bicontinuous microemulsion comprises ghee. In some cases, the bicontinuous microemulsion comprises solid dehydrated lanolin.

The present disclosure provides a bicontinuous microemulsion comprising: a) an aqueous phase comprising: i) sodium chloride; ii) sea salt; iii) ascorbic acid; and iv) trehalose; and b) a lipid phase comprising i) sphingomyelin; ii) phosphatidylcholine; iii) fish oil; iv) palm oil; and v) olive oil, wherein the osmolarity of the microemulsion is from 270 mOsm to 320 mOsm, and wherein the microemulsion has a pH of from 7.2 to 7.4. In some cases, the ratio of aqueous phase to lipid phase is from about 10:1 to 1:10 (v/v). In some cases, the ratio of aqueous phase to lipid phase is from about 5:1 to 1:5 (v/v). In some cases, the ratio of aqueous phase to lipid phase is from about 3:1 to 1:3 (v/v). In some cases, the bicontinuous microemulsion comprises lactoferrin. In some cases, the ratio of sphingomyelin to phosphatidylcholine is from 4:1 to 1:1 (w/v). In some cases, the ratio of palm oil to olive oil is from 3:1 to 1:3 (v/v). In some cases, the ratio of solid oils and liquid oils to beeswax is from about 4:1:0.1 to 1:1:0.3. In some cases, the ratio of coconut oil and grape seed oil to beeswax is from 4:1:0.1 to 1:1:0.3. In some cases, the ratio of aqueous phase to lipid phase is from about 2.5:1 to 3:1 (v/v). In some cases, the ratio of palm oil and olive oil to fish oil is from 10:1 to 6:1 palm oil+olive oil:fish oil. In some cases, the ratio of solid oils and liquid oils to fish oil is from 10:1 to 6:1. In some cases, the ratio of coconut oil and grape seed oil to fish oil is from 10:1 to 6:1. In some cases, the bicontinuous emulsion is sterile. In some cases, the bicontinuous microemulsion comprises coconut oil. In some cases, the ratio of palm oil/coconut oil and olive oil to fish oil is from 10:1 to 6:1 palm oil+olive oil:fish oil. In some cases, the ratio of coconut oil and grape seed oil to fish oil is from 10:1 to 6:1 coconut oil+grape seed oil:fish oil. In some cases, the ratio of coconut oil to grape seed to fish oil is from 10:1 to 6:1 coconut oil+grape seed oil: fish oil. In some cases, the ratio of coconut oil to grape seed oil is from 3:1 to 1:3 (v/v). In some cases, the ratio of coconut oil to grape seed oil is from 5:1 to 1:5 (v/v). In some cases, the ratio of coconut oil to grape seed oil is from 2.5:1 to 3:1 (v/v).). In some cases, the ratio of coconut oil to grape seed oil is from 10:1 to 1:10 (v/v).

The present disclosure provides a container comprising a bicontinuous microemulsion as described above or elsewhere herein. In some cases, the container is an eyedropper. In some cases, the container is sterile; and the bicontinuous emulsion is sterile. The present disclosure provides a method of reducing evaporation of an aqueous tear film from the surface of an eye, the method comprising applying a bicontinuous microemulsion, as described above or elsewhere herein, to the surface of the eye, thereby reducing evaporation of the aqueous tear film from the surface of the eye. The present disclosure provides a method of treating dry eye in an eye of an individual, the method comprising applying a bicontinuous microemulsion, as described above or elsewhere herein, to the surface of the eye of the individual.

Aspects, including embodiments, of the present subject matter described above may be beneficial alone or in combination, with one or more other aspects or embodiments. Without limiting the foregoing description, certain non-limiting aspects of the disclosure numbered 1-32 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. A bicontinuous microemulsion comprising: a) an aqueous phase comprising one or more of: i) sodium chloride; ii) sea salt; iii) ascorbic acid; and iv) trehalose; and b) a lipid phase comprising one or more of: i) sphingomyelin; ii) phosphatidylcholine; iii) fish oil; iv) palm oil; v) olive oil; vi) coconut oil; vii) grapeseed oil; viii) bees wax; xi) ghee; and x) lanolin, wherein the osmolarity of the microemulsion is from 270 mOsm to 320 mOsm, and wherein the microemulsion has a pH of from 7.2 to 7.4.

Aspect 2. The bicontinuous microemulsion of aspect 1, wherein the ratio of aqueous phase to lipid phase is from about 10:1 to 1:10 (v/v).

Aspect 3. The bicontinuous microemulsion of aspect 1, wherein the ratio of aqueous phase to lipid phase is from about 5:1 to 1:5 (v/v).

Aspect 4. The bicontinuous microemulsion of aspect 1, wherein the ratio of aqueous phase to lipid phase is from about 3:1 to 1:3 (v/v).

Aspect 5. The bicontinuous microemulsion of aspect 1, wherein the ratio of aqueous phase to lipid phase is from about 2.5:1 to 3:1 (v/v).

Aspect 6. The bicontinuous microemulsion of any one of aspects 1-5, comprising lactoferrin.

Aspect 7. The bicontinuous microemulsion of any one of aspects 1-6, wherein the lipid phase comprises sphingomyelin and phosphatidylcholine.

Aspect 8. The bicontinuous microemulsion of aspect 7, wherein the ratio of sphingomyelin to phosphatidylcholine is from 4:1 to 1:1 (w/v).

Aspect 9. The bicontinuous microemulsion of any one of aspects 1-6, wherein the lipid phase comprises palm oil and olive oil.

Aspect 10. The bicontinuous microemulsion of aspect 9, wherein the ratio of palm oil to olive oil is from 3:1 to 1:3 (v/v).

Aspect 11. The bicontinuous microemulsion of any one of aspects 1-6, wherein the lipid phase comprises palm oil, olive oil, and fish oil.

Aspect 12. The bicontinuous microemulsion of aspect 11, wherein the ratio of palm oil and olive oil to fish oil is from 10:1 to 6:1 palm oil+olive oil:fish oil.

Aspect 13. The bicontinuous microemulsion of any one of aspects 1-7, comprising coconut oil.

Aspect 14. The bicontinuous microemulsion of any one of aspects 1-7, wherein the lipid phase comprises coconut oil or palm oil; olive oil; and fish oil.

Aspect 15. The bicontinuous microemulsion of aspect 14, wherein the ratio of palm oil/coconut oil and olive oil to fish oil is from 10:1 to 6:1 palm oil/coconut oil+olive oil:fish oil.

Aspect 16. The bicontinuous microemulsion of any one of aspects 1-7, wherein the lipid phase comprises nonpolar lipids and polar lipids in a ratio of from about 10:1 to 4:1 (v/v).

Aspect 17. The bicontinuous microemulsion of any one of aspects 1-7, wherein the lipid phase comprises grapeseed oil, coconut oil, beeswax, sphingomyelin, and phosphatidylcholine.

Aspect 18. The bicontinuous microemulsion of any one of aspects 1-7, wherein the lipid phase comprises palm oil, beeswax, lecithin, and phosphatidylcholine.

Aspect 19. The bicontinuous microemulsion of any one of aspects 1-7, wherein the lipid phase comprises ghee, lecithin, and phosphatidylcholine.

Aspect 20. The bicontinuous microemulsion of any one of aspects 1-7, wherein the lipid phase comprises ghee, lecithin, sphingomyelin, and phosphatidylcholine.

Aspect 21. The bicontinuous microemulsion of any one of aspects 1-7, wherein the lipid phase comprises grapeseed oil, beeswax, lecithin, and sphingomyelin.

Aspect 22. The bicontinuous microemulsion of any one of aspects 1-7, wherein the lipid phase comprises grapeseed oil, coconut oil, beeswax, lecithin, and sphingomyelin.

Aspect 23. The bicontinuous microemulsion of any one of aspects 1-7, wherein the lipid phase comprises ghee, lecithin, and sphingomyelin.

Aspect 24. The bicontinuous microemulsion of any one of aspects 1-7, wherein the lipid phase comprises coconut oil and grapeseed oil.

Aspect 25. The bicontinuous microemulsion of aspect 1, wherein the lecithin is egg yolk lecithin.

Aspect 26. The bicontinuous microemulsion of any one of aspects 1-25, wherein the bicontinuous emulsion is sterile.

Aspect 27. A formulation comprising the bicontinuous microemulsion of any one of aspects 1-26, wherein the formulation is a liquid, a gel, a semi-solid, or an ointment.

Aspect 28. A container comprising the bicontinuous microemulsion of any one of aspects 1-26 or the formulation of aspect 27.

Aspect 30. The container of aspect 28, wherein the container comprises an eyedropper.

Aspect 31. A method of reducing evaporation of an aqueous tear film from the surface of an eye, the method comprising applying the bicontinuous microemulsion of any one of aspects 1-26 or the formulation of aspect 27 to the surface of the eye, thereby reducing evaporation of the aqueous tear film from the surface of the eye.

Aspect 32. A method of treating dry eye in an eye of an individual, the method comprising applying the bicontinuous microemulsion of any one of aspects 1-26 or the formulation of aspect 27 to the surface of the eye of the individual.

### BRIEF DESCRIPTION OF THE DRAWINGS

FIG. 1 depicts surface pressure as a function of model tear lipid (MTL) film thickness for three different MTL mixtures.

FIG. 2 depicts sessile drop of model tear electrolytes (MTE) solution evaporation at 35° C., 75% relative humidity (RH) without lipid films and with ˜90-nm thick model lipid films.

FIG. 3 depicts MTE solution evaporative flux at 35.5° C., 75% RH without lipid films and with model lipid films of varying thickness.

FIG. 4 depicts the surface pressure (in mN/m) of a microemulsion of the present disclosure and human tear lipid, of various thicknesses.

FIG. 5 depicts the relative evaporation rate (as a percent of MTE) of: 1) various model systems; 2) a bicontinuous microemulsion of the present disclosure; and 3) human tears.

FIG. 6 depicts tear-lipid-film quality in a control eye and a test eye (1 second after blink) 2 hours after administration of a bicontinuous microemulsion of the present disclosure.

FIG. 7 depicts tear-lipid-film quality in a control eye and a test eye (6 seconds after blink) 2 hours after administration of a bicontinuous microemulsion of the present disclosure.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a bicontinuous microemulsion” includes a plurality of such microemulsions and reference to “the ocular disorder” includes reference to one or more ocular disorders and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides bicontinuous microemulsions, and methods of using the bicontinuous microemulsions in the treatment of dry eye.

### Microemulsions

The present disclosure provides bicontinuous microemulsions for use in treating dry eye. A bicontinuous microemulsion of the present disclosure comprises: a) an aqueous phase; and b) a lipid phase. The oils and lipids blended in a bicontinuous microemulsion of the present disclosure include only naturally-occurring components that are chemically identical to counterpart components found in human tear lipids. Because a bicontinuous microemulsion of the present disclosure comprises components found in human tear lipids, the lipid phase and the aqueous phase of a bicontinuous microemulsion of the present disclosure are spontaneously arranged into nanometer-sized aggregates possessing the structure and dimensions similar to that of human tear-lipid films.

A bicontinuous microemulsion of the present disclosure comprises droplets having an average diameter in the range of from 25 nm to 200 nm, or from 50 nm to 150 nm.

The present disclosure provides a bicontinuous microemulsion comprising: a) an aqueous phase comprising: i) sodium chloride; ii) sea salt; iii) ascorbic acid; and iv) trehalose; and b) a lipid phase comprising one or more of: i) sphingomyelin; ii) phosphatidylcholine; iii) fish oil; iv) palm oil; v) olive oil; vi) coconut oil; vii) grapeseed oil; viii) beeswax; ix) ghee; and x) solid dehydrated lanolin, wherein the osmolarity of the microemulsion is from 270 mOsm to 320 mOsm, and wherein the microemulsion has a pH of from 7.2 to 7.4. In some cases, the ratio of aqueous phase to lipid phase is from about 10:1 to 1:10 (v/v). In some cases, the ratio of aqueous phase to lipid phase is from about 9:1 to 1:9 (v/v). In some cases, the ratio of aqueous phase to lipid phase is from about 5:1 to 1:5 (v/v). In some cases, the ratio of aqueous phase to lipid phase is from about 3:1 to 1:3 (v/v). In some cases, the ratio of aqueous phase to lipid phase is from about 2.5:1 to 3:1 (v/v).

In some cases, the bicontinuous microemulsion further comprises beeswax. In some cases, the bicontinuous microemulsion comprises palm oil. In some cases, the bicontinuous microemulsion comprises olive oil. In some cases, the bicontinuous microemulsion comprises palm oil and olive oil. In some cases, the bicontinuous microemulsion comprises coconut oil. In some cases, the bicontinuous microemulsion comprises solid dehydrated lanolin. In some cases, the bicontinuous microemulsion comprises coconut oil and grape seed oil. In some cases, the bicontinuous microemulsion further comprises coconut oil, grape seed oil, and beeswax. In some cases, the bicontinuous microemulsion comprises ghee (i.e. clarified butter). In some cases, the bicontinuous microemulsion comprises solid dehydrated lanolin.

In some cases, the bicontinuous emulsion is sterile. The present disclosure provides a container comprising a bicontinuous microemulsion as described above or elsewhere herein. In some cases, the container is an eyedropper. In some cases, the bicontinuous microemulsion formulation is filled into an eyedropper bottle. In some cases, the bicontinuous microemulsion is packaged in an eyedropper bottle with a screw-top dropper. In some cases, the container is sterile; and the bicontinuous emulsion is sterile.

The pH of a bicontinuous microemulsion of the present disclosure is in the range of from about 7.0 to 7.5, from about 7.0 to 7.3, from about 7.1 to about 7.4, from about 7.2 to about 7.5, or from 7.3 to 7.4.

A bicontinuous microemulsion of the present disclosure has an osmolarity of from 200 mOsm to 400 mOsm, from 220 mOsm to 380 mOsm, from 240 mOsm to 360 mOsm, from 260 mOsm to 340 mOsm, or from 270 mOsm to 320 mOsm.

A bicontinuous microemulsion of the present disclosure can form a lipid film on an eye, where the lipid film has a thickness of from 30 nm to 100 nm, e.g., from 30 nm to 50 nm, from 50 nm to 60 nm, from 60 nm to 70 nm, from 70 nm to 80 nm, from 80 nm to 90 nm, or from 90 nm to 100 nm.

A bicontinuous microemulsion of the present disclosure does not contain mineral oil. A bicontinuous microemulsion of the present disclosure does not contain white petrolatum. A bicontinuous microemulsion of the present disclosure does not contain crude oil-derived organic solvents or petroleum-derived organic solvents.

A bicontinuous microemulsion of the present disclosure comprises an aqueous phase and a lipid phase. In some cases, a bicontinuous microemulsion of the present disclosure comprises an aqueous phase and a lipid phase in a ratio of from 10:1 (v/v) to 1:10 (v/v) aqueous phase:lipid phase. In some cases, a bicontinuous microemulsion of the present disclosure comprises an aqueous phase and a lipid phase in a ratio of from 5:1 (v/v) to 1:5 (v/v) aqueous phase:lipid phase. In some cases, a bicontinuous microemulsion of the present disclosure comprises an aqueous phase and a lipid phase in a ratio of from 3:1 (v/v) to 1:3 (v/v) aqueous phase:lipid phase. In some cases, a bicontinuous microemulsion of the present disclosure comprises an aqueous phase and a lipid phase in a ratio of from 2:1 (v/v) to 1:2 (v/v) aqueous phase:lipid phase. In some cases, a bicontinuous microemulsion of the present disclosure comprises an aqueous phase and a lipid phase in a ratio of 3:1 (v/v) aqueous phase:lipid phase. In some cases, a bicontinuous microemulsion of the present disclosure comprises an aqueous phase and a lipid phase in a ratio of 2.5:1 (v/v) aqueous phase:lipid phase. In some cases, a bicontinuous microemulsion of the present disclosure comprises an aqueous phase and a lipid phase in a ratio of 2:1 (v/v) aqueous phase:lipid phase. In some cases, a bicontinuous microemulsion of the present disclosure comprises an aqueous phase and a lipid phase in a ratio of 1:1 (v/v) aqueous phase:lipid phase. In some cases, a bicontinuous microemulsion of the present disclosure comprises an aqueous phase and a lipid phase in a ratio of 1:1.5 (v/v) aqueous phase:lipid phase. In some cases, a bicontinuous microemulsion of the present disclosure comprises an aqueous phase and a lipid phase in a ratio of 1:2 (v/v) aqueous phase:lipid phase. In some cases, a bicontinuous microemulsion of the present disclosure comprises an aqueous phase and a lipid phase in a ratio of 1:2.5 (v/v) aqueous phase:lipid phase. In some cases, a bicontinuous microemulsion of the present disclosure comprises an aqueous phase and a lipid phase in a ratio of 1:3 (v/v) aqueous phase:lipid phase.

### Aqueous Phase

The aqueous phase of a bicontinuous microemulsion of the present disclosure comprises one or more of: a salt, a buffering agent, an anti-oxidant, and an aqueous phase thickener. In some cases, the aqueous phase of a bicontinuous microemulsion of the present disclosure comprises a salt and a buffering agent. In some cases, the aqueous phase of a bicontinuous microemulsion of the present disclosure comprises salt, a buffering agent, and an anti-oxidant. In some cases, the aqueous phase of a bicontinuous microemulsion of the present disclosure comprises salt, a buffering agent, an anti-oxidant, and a thickener.

As noted above, in some cases, the aqueous phase of a bicontinuous microemulsion of the present disclosure comprises a salt. In some cases, the salt is sodium chloride. In some cases, the salt is sea salt. In some cases, the aqueous phase of a bicontinuous microemulsion of the present disclosure includes both sodium chloride and sea salt. In some cases, a bicontinuous microemulsion of the present disclosure comprises 0.5% (w/w) sodium chloride and 0.4% (w/w) sea salt.

As noted above, in some cases, the aqueous phase of a bicontinuous microemulsion of the present disclosure comprises an anti-oxidant. In some cases, the anti-oxidant is ascorbic acid. In some cases, the anti-oxidant is folic acid. In some cases, the anti-oxidant is vitamin E. In some cases, the anti-oxidant is citric acid. In some cases, the anti-oxidant is beta carotene. In some cases, the anti-oxidant is glutathione.

As noted above, in some cases, the aqueous phase of a bicontinuous microemulsion of the present disclosure comprises a buffering agent. In some cases, the buffering agent is ascorbic acid. In some cases, the buffering agent is citric acid. In some cases, the buffering agent is glycolic acid. In some cases, the buffering agent is glycine.

As noted above, in some cases, the aqueous phase of a bicontinuous microemulsion of the present disclosure comprises an aqueous phase thickener. In some cases, the aqueous phase thickener is monosaccharide. In some cases, the aqueous phase thickener is a glucose. In some cases, the aqueous phase thickener is a trehalose. In some cases, the aqueous phase thickener is disaccharide. In some cases, the aqueous phase thickener is lactose. In some cases, the aqueous phase thickener is maltose. In some cases, the aqueous phase thickener is galactose. In some cases, the aqueous phase thickener is mannose. In some cases, the aqueous phase thickener is polysaccharide. In some cases, the aqueous phase thickener is dextran. In some cases, the aqueous phase thickener is agarose. In some cases, the aqueous phase thickener is starch. In some cases, the aqueous phase thickener is guar gum.

In some cases, the aqueous phase of a bicontinuous microemulsion of the present disclosure comprises: i) sodium chloride; ii) sea salt; iii) ascorbic acid; and iv) trehalose. In some cases, a bicontinuous microemulsion of the present disclosure comprises: i) sodium chloride 0.5% (w/w); ii) sea salt 0.4% (w/w); iii) ascorbic acid; and iv) trehalose. In some cases, the aqueous phase of a bicontinuous microemulsion of the present disclosure comprises: i) sodium chloride (5%); ii) sea salt (4%), buffered to pH 7.0 to 7.5 with ascorbic acid, citric acid, or NaH₂PO₄. In some cases, the aqueous phase includes lactoferrin. In some cases, the aqueous phase includes mucin. In some cases, the aqueous phase includes alginate. In some cases, the aqueous phase includes a polysaccharide.

### Lipid Phase

The lipid phase of a bicontinuous microemulsion of the present disclosure comprises one or more lipids that are naturally present in human tears.

The lipid phase of a bicontinuous microemulsion of the present disclosure comprises one or more of: i) sphingomyelin; ii) phosphatidylcholine; iii) fish oil; iv) palm oil; v) olive oil; vi) coconut oil; vii) grapeseed oil; viii) bees wax; xi) ghee; and x) lanolin. In some cases, a bicontinuous microemulsion of the present disclosure specifically excludes one or more of: i) sphingomyelin; ii) phosphatidylcholine; iii) fish oil; iv) palm oil; v) olive oil; vi) coconut oil; vii) grapeseed oil; viii) bees wax; xi) ghee; and x) lanolin. For example, in some cases, a bicontinuous microemulsion of the present disclosure specifically excludes palm oil.

In some cases, the lipid phase of a bicontinuous microemulsion of the present disclosure comprises sphingomyelin. In some cases, the lipid phase of a bicontinuous microemulsion of the present disclosure comprises phosphatidylcholine (lecithin). In some cases, the lipid phase of a bicontinuous microemulsion of the present disclosure comprises sphingomyelin and phosphatidylcholine. The sphingomyelin and lecithin can be from any of a variety of sources, which may include cow milk, egg yolk, beef liver, porcine brain, and soy. In some cases, the lecithin is egg yolk lecithin. In some cases, the sphingomyelin is egg yolk sphingomyelin. In some cases, the lipid phase of a bicontinuous microemulsion of the present disclosure comprises fish oil. In some cases, the lipid phase of a bicontinuous microemulsion of the present disclosure comprises palm oil. In some cases, the lipid phase of a bicontinuous microemulsion of the present disclosure comprises olive oil. In some cases, the lipid phase of a bicontinuous microemulsion of the present disclosure comprises coconut oil. In some cases, the lipid phase of a bicontinuous microemulsion of the present disclosure comprises grape seed oil. In some cases, the lipid phase of a bicontinuous microemulsion of the present disclosure comprises beeswax. In some cases, the lipid phase of a bicontinuous microemulsion of the present disclosure comprises ghee. In some cases, the lipid phase of a bicontinuous microemulsion of the present disclosure comprises palm oil and olive oil. In some cases, the lipid phase of a bicontinuous microemulsion of the present disclosure comprises coconut oil and grape seed oil. In some cases, the lipid phase of a bicontinuous microemulsion of the present disclosure comprises coconut oil, grape seed oil, and beeswax. In some cases, the lipid phase of a bicontinuous microemulsion of the present disclosure comprises ghee, beeswax and fish oil. In some cases, the lipid phase of a bicontinuous microemulsion of the present disclosure comprises palm oil, olive oil, and fish oil. In some cases, the lipid phase of a bicontinuous microemulsion of the present disclosure comprises sphingomyelin, phosphatidylcholine, fish oil, palm oil, and olive oil. In some cases, the lipid phase of a bicontinuous microemulsion of the present disclosure comprises sphingomyelin, phosphatidylcholine, fish oil, coconut oil, and grape seed oil. In some cases, the lipid phase of a bicontinuous microemulsion of the present disclosure comprises sphingomyelin, phosphatidylcholine, fish oil, palm oil, and grape seed oil.

The lipid phase of a bicontinuous microemulsion of the present disclosure can comprise non-polar lipids and polar lipids. In some cases, the ratio of non-polar lipids to polar lipids in the lipid phase is from 10:1 to 4:1, e.g., from 10:1 to 8:1, from 8:1 to 6:1, or from 6:1 to 4:1.

As noted above, in some cases, the lipid phase of a bicontinuous microemulsion of the present disclosure comprises sphingomyelin. The sphingomyelin can be present in the microemulsion in an amount of from 5% to 25% by weight, from 10% to 20% by weight, or from 10% to 15% by weight. As noted above, in some cases, the lipid phase of a bicontinuous microemulsion of the present disclosure comprises phosphatidylcholine. The phosphatidylcholine can be present in the microemulsion in an amount of from 5% to 25% by weight, from 10% to 20% by weight, or from 10% to 15% by weight.

As noted above, in some cases, the lipid phase of a bicontinuous microemulsion of the present disclosure comprises sphingomyelin and phosphatidylcholine. In some cases, a bicontinuous microemulsion of the present disclosure comprises sphingomyelin and phosphatidylcholine in a ratio of from 4:1 to 1:1 sphingomyelin:phosphatidylcholine. In some cases, a bicontinuous microemulsion of the present disclosure comprises sphingomyelin and phosphatidylcholine in a ratio of from 3:1 to 1:1 sphingomyelin:phosphatidylcholine. In some cases, a bicontinuous microemulsion of the present disclosure comprises sphingomyelin and phosphatidylcholine in a ratio of from 2:1 to 1:1 sphingomyelin:phosphatidylcholine. In some cases, a bicontinuous microemulsion of the present disclosure comprises sphingomyelin and phosphatidylcholine in a ratio of from 3:1 to 2:1 sphingomyelin:phosphatidylcholine. In some cases, a bicontinuous microemulsion of the present disclosure comprises sphingomyelin and phosphatidylcholine in a ratio of 4:1 sphingomyelin:phosphatidylcholine. In some cases, a bicontinuous microemulsion of the present disclosure comprises sphingomyelin and phosphatidylcholine in a ratio of 3:1 sphingomyelin:phosphatidylcholine. In some cases, a bicontinuous microemulsion of the present disclosure comprises sphingomyelin and phosphatidylcholine in a ratio of 2:1 sphingomyelin:phosphatidylcholine. In some cases, a bicontinuous microemulsion of the present disclosure comprises sphingomyelin and phosphatidylcholine in a ratio of 1:1 sphingomyelin:phosphatidylcholine.

As noted above, in some cases, the lipid phase of a bicontinuous microemulsion of the present disclosure comprises palm oil and olive oil. In some cases, the palm oil and the olive oil are present in a bicontinuous microemulsion of the present disclosure in a ratio of from 1:3 to 3:1 palm oil:olive oil. In some cases, the palm oil and the olive oil are present in a bicontinuous microemulsion of the present disclosure in a ratio of 1:3 palm oil:olive oil. In some cases, the palm oil and the olive oil are present in a bicontinuous microemulsion of the present disclosure in a ratio of 1:2 palm oil:olive oil. In some cases, the palm oil and the olive oil are present in a bicontinuous microemulsion of the present disclosure in a ratio of 1:1 palm oil:olive oil. In some cases, the palm oil and the olive oil are present in a bicontinuous microemulsion of the present disclosure in a ratio of 2:1 palm oil:olive oil. In some cases, the palm oil and the olive oil are present in a bicontinuous microemulsion of the present disclosure in a ratio of 3:1 palm oil:olive oil.

As noted above, in some cases, the lipid phase of a bicontinuous microemulsion of the present disclosure comprises coconut oil and grape seed oil. In some cases, the coconut oil and the grape seed oil are present in a bicontinuous microemulsion of the present disclosure in a ratio of from 10:1 to 1:10 coconut oil:grape seed oil. In some cases, the coconut oil and the grape seed oil are present in a bicontinuous microemulsion of the present disclosure in a ratio of from 1:3 to 3:1 coconut oil:grape seed oil. In some cases, the coconut oil and the grape seed oil are present in a bicontinuous microemulsion of the present disclosure in a ratio of 1:3 coconut oil:grape seed oil. In some cases, the coconut oil and the grape seed oil are present in a bicontinuous microemulsion of the present disclosure in a ratio of 1:2 coconut oil:grape seed oil. In some cases, the coconut oil and the grape seed oil are present in a bicontinuous microemulsion of the present disclosure in a ratio of 1:1 coconut oil: grape seed oil. In some cases, the coconut oil and the grape seed oil are present in a bicontinuous microemulsion of the present disclosure in a ratio of 2:1 coconut oil:grape seed oil. In some cases, the coconut oil and the grape seed oil are present in a bicontinuous microemulsion of the present disclosure in a ratio of 3:1 coconut oil oil:grape seed oil.

Grapeseed oil can comprise about 60% to 80% linoleic acid; from 14% to 16% oleic acid; from 5% to 8% palmitic acid; from 4% to 5% stearic acid; and from about 0.05% to 0.5% alpha-linolenic acid. See, e.g., Moret at al. (2000) J. American Oil Chemists' Society 77:1289.

Beeswax comprises fatty acid esters and various long-chain alcohols. For example, beeswax can comprise palmitate, palmitoleate, and oleate esters of long-chain (e.g., from 30 to 32 carbons) aliphatic alcohols. Principle components can be triacontanyl palmitate and cerotic acid, which can be present in a ratio of 6:1 triacontanyl palmitate:cerotic acid. See, e.g., Tulloch (1970) Lipids 5:247.

As noted above, in some cases, the lipid phase of a bicontinuous microemulsion of the present disclosure comprises palm oil and fish oil. In some cases, the palm oil and the fish oil are present in a bicontinuous microemulsion of the present disclosure in a ratio of from 8:1 to 6:1 palm oil:fish oil. In some cases, the palm oil and the fish oil are present in a bicontinuous microemulsion of the present disclosure in a ratio of 8:1 palm oil:fish oil. In some cases, the palm oil and the fish oil are present in a bicontinuous microemulsion of the present disclosure in a ratio of 7:1 palm oil:fish oil. In some cases, the palm oil and the fish oil are present in a bicontinuous microemulsion of the present disclosure in a ratio of 6:1 palm oil:fish oil.

As noted above, in some cases, the lipid phase of a bicontinuous microemulsion of the present disclosure comprises coconut oil and fish oil. In some cases, the coconut oil and the fish oil are present in a bicontinuous microemulsion of the present disclosure in a ratio of from 8:1 to 6:1 palm oil:fish oil. In some cases, the coconut oil and the fish oil are present in a bicontinuous microemulsion of the present disclosure in a ratio of 8:1 coconut oil:fish oil. In some cases, the coconut oil and the fish oil are present in a bicontinuous microemulsion of the present disclosure in a ratio of 7:1 coconut oil:fish oil. In some cases, the coconut oil and the fish oil are present in a bicontinuous microemulsion of the present disclosure in a ratio of 6:1 coconut oil:fish oil.

As noted above, in some cases, the lipid phase of a bicontinuous microemulsion of the present disclosure comprises palm oil, olive oil, and fish oil. In some cases, the palm oil, olive oil, and fish oil are present in a bicontinuous microemulsion of the present disclosure in a ratio of from 10:1 to 8:1 (palm oil+olive oil):fish oil. As noted above, in some cases, the lipid phase of a bicontinuous microemulsion of the present disclosure comprises palm oil, olive oil, and fish oil. In some cases, the palm oil, olive oil, and fish oil are present in a bicontinuous microemulsion of the present disclosure in a ratio of 10:1 (palm oil+olive oil):fish oil. In some cases, the palm oil, olive oil, and fish oil are present in a bicontinuous microemulsion of the present disclosure in a ratio of 9:1 (palm oil+olive oil):fish oil. In some cases, the palm oil, olive oil, and fish oil are present in a bicontinuous microemulsion of the present disclosure in a ratio of 8:1 (palm oil+olive oil):fish oil.

As noted above, in some cases, the lipid phase of a bicontinuous microemulsion of the present disclosure comprises coconut oil, olive oil, and fish oil. In some cases, the coconut oil, olive oil, and fish oil are present in a bicontinuous microemulsion of the present disclosure in a ratio of from 10:1 to 8:1 (coconut oil+olive oil):fish oil. As noted above, in some cases, the lipid phase of a bicontinuous microemulsion of the present disclosure comprises coconut oil, olive oil, and fish oil. In some cases, the coconut oil, olive oil, and fish oil are present in a bicontinuous microemulsion of the present disclosure in a ratio of 10:1 (coconut oil+olive oil):fish oil. In some cases, the coconut oil, olive oil, and fish oil are present in a bicontinuous microemulsion of the present disclosure in a ratio of 9:1 (coconut oil+olive oil):fish oil. In some cases, the coconut oil, olive oil, and fish oil are present in a bicontinuous microemulsion of the present disclosure in a ratio of 8:1 (coconut oil+olive oil):fish oil.

In some cases, the lipid phase thickener is coconut oil. In some cases, the lipid phase contains palm oil, where the palm oil is a source of beta-carotenes.

Phosphatidylcholines include, e.g., L-α phosphatidylcholines; 1-oleoyl-2-palmitoyl-phosphatidylcholine; and the like. Lecithin can comprise phospholipids such as phosphatidylcholine, phosphatidylethanolamine, phosphatidylinositol, phosphatidylserine, and phosphatidic acid. In some cases, the lecithin included in a bicontinuous microemulsion of the present disclosure includes multiple forms of lecithin. Sphingomyelin, lecithin, and phosphatidylcholine that may be included in a bicontinuous microemulsion of the present disclosure can be heterogeneous mixtures. Sphingomyelin, lecithin, and phosphatidylcholine that may be included in a bicontinuous microemulsion of the present disclosure can be purified, e.g., from 50% to 60%, from 60% to 70%, from 70% to 80%, from 80% to 90%, from 90% to 95%, from 95% to 98%, or more than 98%, pure.

In some cases, the lipid phase of a bicontinuous microemulsion of the present disclosure comprises palm oil, olive oil, and flax seed oil. In some cases, the lipid phase of a bicontinuous microemulsion of the present disclosure comprises palm oil, olive oil, and grape seed oil (as a source of omega-3,6 and 9 fatty acids). In some cases, the lipid phase of a bicontinuous microemulsion of the present disclosure comprises palm oil, olive oil, and Sea Buckthorn oil (Omega-7, palmitoleic and cis-vaccenic acids).

As noted above, in some cases, the lipid phase of a bicontinuous microemulsion of the present disclosure comprises palm oil, grape seed oil, and fish oil. In some cases, the palm oil, grape seed oil, and fish oil are present in a bicontinuous microemulsion of the present disclosure in a ratio of from 10:1 to 8:1 (palm oil+grape seed oil):fish oil. As noted above, in some cases, the lipid phase of a bicontinuous microemulsion of the present disclosure comprises palm oil, grape seed oil, and fish oil. In some cases, the palm oil, grape seed oil, and fish oil are present in a bicontinuous microemulsion of the present disclosure in a ratio of 10:1 (palm oil+grape seed oil):fish oil. In some cases, the palm oil, grape seed oil, and fish oil are present in a bicontinuous microemulsion of the present disclosure in a ratio of 9:1 (palm oil+grape seed oil):fish oil. In some cases, the palm oil, grape seed oil, and fish oil are present in a bicontinuous microemulsion of the present disclosure in a ratio of 8:1 (palm oil+grape seed oil):fish oil.

As noted above, in some cases, the lipid phase of a bicontinuous microemulsion of the present disclosure comprises coconut oil, grape seed oil, and fish oil. In some cases, the coconut oil, grape seed oil, and fish oil are present in a bicontinuous microemulsion of the present disclosure in a ratio of from 10:1 to 8:1 (coconut oil+grape seed oil):fish oil. In some cases, the coconut oil, grape seed oil, and fish oil are present in a bicontinuous microemulsion of the present disclosure in a ratio of from 10:1 to 3:1 (coconut oil+grape seed oil):fish oil. In some cases, the ratio of solid oils and liquid oils to fish oil is from 10:1 to 6:1. In some cases, the ratio of coconut oil and grape seed oil to fish oil is from 10:1 to 6:1. As noted above, in some cases, the lipid phase of a bicontinuous microemulsion of the present disclosure comprises coconut oil, grape seed oil, and fish oil. In some cases, the coconut oil, grape seed oil, and fish oil are present in a bicontinuous microemulsion of the present disclosure in a ratio of 10:1 (coconut oil+grape seed oil):fish oil. In some cases, the coconut oil, grape seed oil, and fish oil are present in a bicontinuous microemulsion of the present disclosure in a ratio of 9:1 (coconut oil+grape seed oil):fish oil. In some cases, the coconut oil, grape seed oil, and fish oil are present in a bicontinuous microemulsion of the present disclosure in a ratio of 8:1 (coconut oil+grape seed oil):fish oil. In some cases, the coconut oil, olive oil, and fish oil are present in a bicontinuous microemulsion of the present disclosure in a ratio of 7:1 (coconut oil+olive oil):fish oil. In some cases, the coconut oil, olive oil, and fish oil are present in a bicontinuous microemulsion of the present disclosure in a ratio of 6:1 (coconut oil+grape seed oil):fish oil. In some cases, the coconut oil, grape seed oil, and fish oil are present in a bicontinuous microemulsion of the present disclosure in a ratio of 5:1 (coconut oil+grape seed oil):fish oil. In some cases, the coconut oil, grape seed oil, and fish oil are present in a bicontinuous microemulsion of the present disclosure in a ratio of 4:1 (coconut oil+grape seed oil):fish oil.

In some cases, the coconut oil, grape seed oil, and fish oil are present in a bicontinuous microemulsion of the present disclosure in a ratio of 3:1 (coconut oil+grape seed oil):fish oil.

As noted above, in some cases, the lipid phase of a bicontinuous microemulsion of the present disclosure comprises coconut oil, grape seed oil, and beeswax. In some cases, the coconut oil, grape seed oil, and beeswax are present in a bicontinuous microemulsion of the present disclosure in a ratio of from 10:1 to 8:1 (coconut oil+grape seed oil): beeswax. In some cases, the coconut oil, grape seed oil, and beeswax are present in a bicontinuous microemulsion of the present disclosure in a ratio of from 10:1 to 3:1 (coconut oil+grape seed oil): beeswax. In some cases, the ratio of solid oils and liquid oils to beeswax is from about 4:1:0.1 to 1:1:0.3. In some cases, the ratio of coconut oil and grape seed oil to bees wax is from 4:1:0.1 to 1:1:0.3.

As noted above, in some cases, the lipid phase of a bicontinuous microemulsion of the present disclosure comprises coconut oil, grape seed oil, and bees wax. In some cases, the coconut oil, grape seed oil, and beeswax are present in a bicontinuous microemulsion of the present disclosure in a ratio of 10:1 (coconut oil+grape seed oil): bees wax. In some cases, the coconut oil, grape seed oil, and beeswax are present in a bicontinuous microemulsion of the present disclosure in a ratio of 9:1 (coconut oil+grape seed oil): beeswax. In some cases, the coconut oil, grape seed oil, and beeswax are present in a bicontinuous microemulsion of the present disclosure in a ratio of 8:1 (coconut oil+grape seed oil): beeswax. In some cases, the coconut oil, grape seed oil, and beeswax are present in a bicontinuous microemulsion of the present disclosure in a ratio of 7:1 (coconut oil+grape seed oil): beeswax. In some cases, the coconut oil, grape seed oil, and beeswax are present in a bicontinuous microemulsion of the present disclosure in a ratio of 6:1 (coconut oil+grape seed oil): beeswax. In some cases, the coconut oil, grape seed oil, and beeswax are present in a bicontinuous microemulsion of the present disclosure in a ratio of 5:1 (coconut oil+grape seed oil): beeswax. In some cases, the coconut oil, grape seed oil, and beeswax are present in a bicontinuous microemulsion of the present disclosure in a ratio of 4:1 (coconut oil+grape seed oil): beeswax. In some cases, the coconut oil, grape seed oil, and beeswax are present in a bicontinuous microemulsion of the present disclosure in a ratio of 3:1 (coconut oil+grape seed oil): beeswax.

### Additional Components

In addition to the above-noted components, a bicontinuous microemulsion of the present disclosure will in some cases comprise one or more additional components. In some cases, a bicontinuous microemulsion of the present disclosure comprises human lactoferrin (e.g., human milk lactoferrin). In some cases, a bicontinuous microemulsion of the present disclosure comprises human lactoferrin (e.g., human milk lactoferrin) in an amount of from 0.01% to 1.0% (w/w), e.g., from 0.01% (w/w) to 0.1% (w/w), from 0.1% (w/w) to 0.5% (w/w), or from 0.5% (w/w) to 1.0% (w/w).

In some cases, the aqueous phase of a bicontinuous microemulsion of present disclosure comprises Niacinamide. In some cases, the aqueous phase of a bicontinuous microemulsion comprises Quercetin. In some cases, the aqueous phase of a bicontinuous microemulsion Lutein. In some cases, the aqueous phase of a bicontinuous microemulsion comprises Zeaxanthin.

### EXEMPLARY FORMULATINS

Provided herein are exemplary bicontinuous microemulsion formulations. In some cases, a bicontinuous microemulsion of the present disclosure is in the form of a solution. In some cases, a bicontinuous microemulsion is in the form of a suspension. In some cases, the suspension is in an aqueous liquid or non-aqueous liquid. Formulations suitable for injectable use include sterile aqueous solutions. In some cases, a bicontinuous microemulsion of the present disclosure is in the form of an ointment. In some cases, the bicontinuous microemulsion is applied to the surface of an eye in the form of a solution (e.g., an emulsion, a suspension) or an ointment (particularly, an ointment for eyes). In some cases, a non-limiting example of how an ointment can be prepared includes using a widely used basal agent such as ghee or beeswax. Petrochemicals such as white Vaseline or liquid paraffin are excluded. In some cases, the solution or ointment is sterile. A bicontinuous emulsion of the present disclosure can be formulated as a liquid, a gel, a semi-solid, a solid, an ointment, and the like; in some cases, the formulation is sterile.

The following are non-limiting examples of bicontinuous microemulsions of the present disclosure and their preparation.

### Example 1

A bicontinuous microemulsion of the present disclosure can be prepared in the following manner Aqueous and lipid phases can be heated to 36-45° C. in separate vials for 20 minutes. The aqueous and lipid phases can then be placed in one vial and homogenized in an ultrasound bath at medium to high intensity until totally emulsified. Prepared emulsions can be stored at 4° C. and brought to room temperature prior to use. In some cases, no phase separation and emulsion creaming occurs during cold storage for at least one month.

Moreover, different formulations can be targeted to specific dry-eye related deficiencies. For example, formulations can be designed for aqueous-deficient and meibomian gland dysfunction (MGD)-symptomatic or lipid-deficient patients.

Table 1, shown below, provides the aqueous and lipid ranges for aqueous-deficient and MGD lipid-deficient symptoms.

TABLE 1

| | Aqueous phase range, | Lipid phase range, |
|---|---|---|
| Symptoms | parts | parts |
| Aqueous-deficient | 9-15 | 1-2.5 |
| MGD | 1-5 | 5-9 |

Microemulsions 2-16 (“Examples 2-16”) are described below. These microemulsions are examples of bicontinuous microemulsions of the present disclosure, and are not meant to be limiting.

### Example 2

Example 2 provides bicontinuous microemulsion formulation 1 consisting of:
1. 8 parts of isotonic aqueous phase, containing 5% of sodium chloride, 4% of sea salt, buffered with NaH₂ PO₄ to pH 7.3
2. 2 parts of Lipid phase, containing 8 parts of nonpolar lipids (1 part of Grapeseed oil, 2 parts of coconut oil, 0.2 parts of beeswax) and 2 parts of polar lipids (4 parts of Sphingomyelin and 6 parts of L-α Phosphatidylcholines).

### Example 3

Example 3 provides bicontinuous microemulsion formulation 2 consisting of 99.9% natural food-quality organic ingredients which includes:
1: 9 parts of isotonic aqueous phase, containing 5% of sodium chloride, 4% of sea salt, buffered with NaH₂ PO₄ to pH 7.3, containing 0.9 mg/ml of human milk lactoferrin and 0.03 mg/ml of Bovine mucin.
2: 1 parts of Lipid phase, containing 8 parts of nonpolar lipids (1 part of organic Grapeseed oil, 3 parts of organic coconut oil, 0.2 parts of organic beeswax) and 2 parts of polar lipids (4 parts of lecithin and 1 part of L-α Phosphatidylcholines). In some cases, the lecithin is egg yolk lecithin.

### Example 4

Example 4 provides bicontinuous microemulsion 3 consisting of 99.9% natural food-quality organic ingredients, which includes:

1. 9 parts of isotonic aqueous phase, containing 5% of sodium chloride, 4% of sea salt, buffered with citric acid to pH 7.3

2. 1 parts of Lipid phase, containing 7.5 parts of nonpolar lipids (1 part of organic Grapeseed oil, 3 parts of organic coconut oil, 0.2 parts of organic beeswax) and 2.5 parts of polar lipids (5 parts of lecithin and 1 parts of L-α Phosphatidylcholines). In some cases, the lecithin is egg yolk lecithin.

### Example 5

Example 5 provides bicontinuous microemulsion 4 consisting of 99.9% natural food-quality organic ingredients, which includes:

1. 9 parts of isotonic aqueous phase, containing 5% of sodium chloride, 4% of sea salt, buffered with ascorbic acid to pH 7.3.

2. 1 parts of Lipid phase, containing 8 parts of nonpolar lipids (1 part of organic Grapeseed oil, 3 parts of organic coconut oil, 0.2 parts of organic beeswax) and 2 parts of polar lipids (5 parts of lecithin and 1 parts of L-α Phosphatidylcholines). In some cases, the lecithin is egg yolk lecithin.

### Example 6

Example 6 provides bicontinuous microemulsion 5 consisting of 99.9% natural food-quality organic ingredients, which includes:

1. 9 parts of isotonic aqueous phase, containing 5% of sodium chloride, 4% of sea salt, buffered with ascorbic acid to pH 7.3.

2. 1 part of Lipid phase, containing 8 parts of nonpolar lipids (9 parts Organic Red palm oil and 1 part of organic beeswax) and 2 parts of polar lipids (2 parts of lecithin and 1 part of L-α Phosphatidylcholines). In some cases, the lecithin is egg yolk lecithin.

### Example 7

Example 7 provides bicontinuous microemulsion 6 consisting of 99.9% natural food-quality organic ingredients, which includes:

1. 9 parts of isotonic aqueous phase, containing 5% of sodium chloride, 4% of sea salt, buffered with ascorbic acid to pH 7.3.

2. 1 part of Lipid phase, containing 8 parts of nonpolar lipids (Organic Ghee) and 2 parts of polar lipids (5 parts of lecithin and 1 part of L-α Phosphatidylcholines). In some cases, the lecithin is egg yolk lecithin.

### Example 8

Example 8 provides bicontinuous microemulsion 7 consisting of 99.9% natural food-quality organic ingredients, which includes:

1. 8 parts of isotonic aqueous phase, containing 5% of sodium chloride, 4% of sea salt, buffered with ascorbic acid to pH 7.3.

2. 2 parts of Lipid phase, containing 8 parts of nonpolar lipids (Organic Ghee) and 2 parts of polar lipids (4 parts of lecithin, 1 part of Sphingomyelin, and 1 part of L-α Phosphatidylcholines). In some cases, the lecithin is egg yolk lecithin. In some cases, the sphingomyelin is egg yolk sphingomyelin.

### Example 9

Example 9 provides bicontinuous microemulsion 8 consisting of 99.9% natural food-quality organic ingredients, which includes:

1. 9 parts of isotonic aqueous phase, containing 5% of sodium chloride, 4% of sea salt, and 3% of Trehalose, buffered with ascorbic acid to pH 7.3.

2. 2 parts of Lipid phase, containing 8 parts of nonpolar lipids (Organic Ghee) and 2 parts of polar lipids (4 parts lecithin, 1 part of Sphingomyelin, and 1 parts of L-α Phosphatidylcholines). In some cases, the lecithin is egg yolk lecithin. In some cases, the sphingomyelin is egg yolk sphingomyelin.

### Example 10

Example 10 provides bicontinuous microemulsion 9 consisting of 99.9% natural food-quality organic ingredients, which includes:

1. 9 parts of isotonic aqueous phase, containing 5% of sodium chloride, 4% of sea salt, 1% of sodium alginate, buffered with citric acid to pH 7.3.

2. 1 parts of Lipid phase, containing 8 parts of nonpolar lipids (Organic Ghee) and 2 parts of polar lipids (4 parts of lecithin, 1 part of Sphingomyelin, and 1 part of L-α Phosphatidylcholines). In some cases, the lecithin is egg yolk lecithin. In some cases, the sphingomyelin is egg yolk sphingomyelin.

### Example 11

Example 11 provides bicontinuous microemulsion 10 consisting of 99.9% natural food-quality organic ingredients, which includes:

1. 9 parts of isotonic aqueous phase, containing 5% of sodium chloride, 4% of sea salt, 0.5% of Manugel (alginate, polysaccharide) buffered with citric acid to pH 7.3.

2. 2 parts of Lipid phase, containing 8 parts of nonpolar lipids (lipids (2 part of organic Grapeseed oil, 2 parts of organic coconut oil, 0.4 parts of organic beeswax)) and 2 parts of polar lipids (5 parts of lecithin, 1 part of Sphingomyelin). In some cases, the lecithin is egg yolk lecithin. In some cases, the sphingomyelin is egg yolk sphingomyelin.

### Example 12

Example 12 provides bicontinuous microemulsion 11 for aqueous-deficiency symptoms consisting of 99.9% natural food-quality organic ingredients, which includes:

1. 9 parts of isotonic aqueous phase, containing 5% of sodium chloride, 4% of sea salt, 0.5% of Manugel (polysaccharide) buffered with citric acid to pH 7.3.

2. 1 parts of Lipid phase, containing 8 parts of nonpolar lipids (2 part of organic Grapeseed oil, 2 parts of organic coconut oil, 0.4 parts of organic beeswax)) and 2 parts of polar lipids (5 parts of lecithin, 1 part of Sphingomyelin). In some cases, the lecithin is egg yolk lecithin. In some cases, the sphingomyelin is egg yolk sphingomyelin.

### Example 13

Example 13 provides bicontinuous microemulsion 12 for MGD lipid-deficiency symptoms consisting of 99.9% natural food-quality organic ingredients:

1. 1 parts of isotonic aqueous phase, containing 5% of sodium chloride, 4% of sea salt, 0.5% of Manugel (polysaccharide) buffered with ascorbic acid to pH 7.3.

2. 4 parts of Lipid phase, containing 8 parts of nonpolar lipids (Ghee) and 2 parts of polar lipids (5 parts of lecithin, 1 part of Sphingomyelin). In some cases, the lecithin is egg yolk lecithin. In some cases, the sphingomyelin is egg yolk sphingomyelin.

### Example 14

Different formulations targeting specific dry-eye related deficiency, namely, specifically designed for patients of Asian and Caucasian decent have been suggested.

Moreover, different formulations targeting specific dry-eye related deficiency were suggested, as shown in Table 2 below:

TABLE 2

| | Aqueous phase | Lipid phase | Including | Including |
|---|---|---|---|---|
| Race | range, parts | range, parts | Nonpolar parts | Polar parts |
| Asian | 1-10 | 9-1 | 8-9 | 2-1 |
| Caucasian | 1-4 | 4-1 | 7-8 | 3-2 |

### Example 15

Example 15 provides bicontinuous microemulsion 13 for Asian subjects consisting of 99.9% natural food-quality organic ingredients, which include:

1. 8 parts of isotonic aqueous phase, containing 5% of sodium chloride, 4% of sea salt, buffered with citric acid to pH 7.3.

2. 2 parts of Lipid phase, containing 8.5 parts of nonpolar lipids (2 parts of organic Grapeseed oil, 2 parts of organic coconut oil, 0.4 parts of organic beeswax)) and 1.5 parts of polar lipids (5 parts of lecithin, 1 part of Sphingomyelin). In some cases, the lecithin is egg yolk lecithin. In some cases, the sphingomyelin is egg yolk sphingomyelin.

### Example 16

Example 16 provides bicontinuous microemulsion 14 for Caucasian subjects. consisting of 99.9% natural food-quality organic ingredients, which include:

1. 9 parts of isotonic aqueous phase, containing 5% of sodium chloride, 4% of sea salt, 0.5% of Manugel (polysaccharide) buffered with citric acid to pH 7.3. Manugel is an alginate, a naturally occurring polysaccharide; suitable sources include, e.g., seaweed.

2. 1 parts of Lipid phase, containing 3 parts of nonpolar lipids (2 part of organic Grapeseed oil, 2 parts of organic coconut oil, 0.4 parts of organic beeswax)) and 1 parts of polar lipids (3 parts of lecithin, 1 part of Sphingomyelin and 1 part of L-α Phosphatidylcholines). In some cases, the lecithin is egg yolk lecithin. In some cases, the sphingomyelin is egg yolk sphingomyelin.

### Containers

The present disclosure provides a container comprising a bicontinuous microemulsion of the present disclosure. In some cases, the container is sterile, and the bicontinuous microemulsion of the present disclosure is sterile. In some cases, the container is an eyedropper.

### Methods

The present disclosure provides methods of treating dry eye by reducing evaporation of an aqueous tear film from the surface of an eye, the methods generally involving administering, to an ocular surface of an individual having dry eye, an effective amount of a bicontinuous microemulsion of the present disclosure to the eye of an individual in need thereof (e.g., to the surface of the eye). The present disclose provides methods of reducing the rate of evaporation of tears from an eye in an individual, e.g., from an eye in a human; the methods generally involve applying an effective amount of a bicontinuous microemulsion of the present disclosure to the surface of the eye of the individual.

In some cases, an effective amount of a bicontinuous microemulsion of the present disclosure is an amount that reduces the evaporation rate of the aqueous tear film from the surface of the eye. In some cases, an effective amount of a bicontinuous microemulsion of the present disclosure is an amount that reduces the evaporation rate of the aqueous tear film from the surface of the eye by at least 25%, at least 30%, at least 40%, at least 50%, at least 60%, at least 75%, at least 2-fold, at least 3-fold, at least 4-fold, at least 5-fold, or more than 5-fold, compared to the evaporation rate in an untreated eye (e.g., an untreated eye of the same individual under the same conditions).

An individual suitable for treatment with a bicontinuous microemulsion of the present disclosure is an individual who is experiencing dry eye. The dry eye can be due to any of a number of factors, including, but not limited to, inadequate tear production attributable to aging, medical procedures performed on the cornea, or other conditions; exposure of the eye to environmental irritants (e.g., dust, smoke, wind, sun, low humidity); and eye strain attributable to extended viewing of computer monitors, or to other workplace environment-related factors. In some cases, the individual is a human.

A bicontinuous microemulsion of the present disclosure can be applied to the surface of an eye in an amount of from 2 μl to 10 μl, e.g., from 2 μl to 3 μl, from 3 μl to 4 μl, from 4 μl to 5 μl, or from 5 μl to 10 μl.

A bicontinuous microemulsion of the present disclosure can be applied to the surface of an eye at any desired frequency, e.g., from 1 time per hour to 12 times per hour, from 1 time per hour to 1 time per every 2 hours, from once every 2 hours to once every 4 hours, from once every 4 hours to once every 8 hours, or from once every 8 hours to once every 12 hours. A bicontinuous microemulsion of the present disclosure can be applied to the surface of an eye at a frequency of from once every 12 hours to once a day, once every 3 days, once a week, etc.

A bicontinuous microemulsion of the present disclosure can be applied to the surface of an eye for any desired duration, e.g., from one hour to 12 hours, from 12 hours to one day, from 1 day to 7 days, from 1 week to 4 weeks, from 1 month to 6 months, from 6 months to 1 year, or longer than 1 year.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

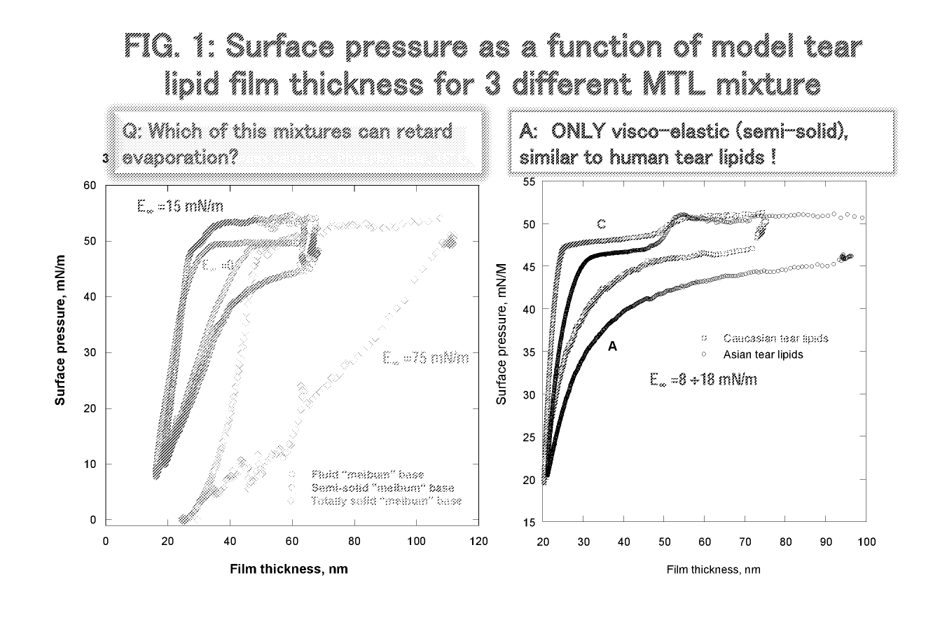

FIG. 1 depicts surface pressure as a function of model tear lipid (MTL) film thickness for three different MTL mixtures.

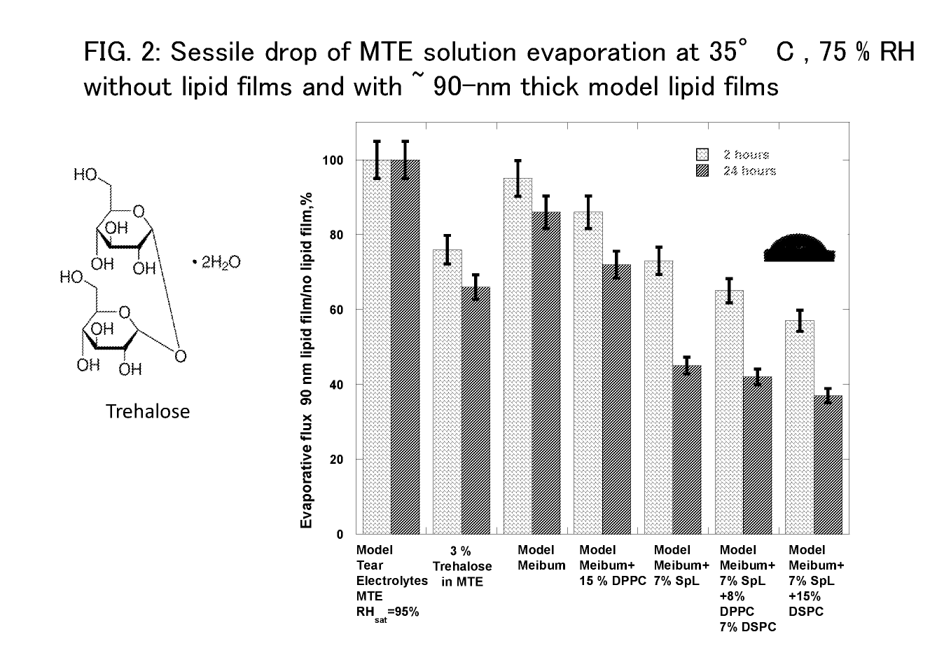

FIG. 2 depicts sessile drop of model tear electrolytes (MTE) solution evaporation at 35° C., 75% RH without lipid films and with ˜90-nm thick model lipid films. DPPC: Dipalmitoylphosphatidylcholine; DSPC: 1,2-distearoyl-sn-glycero-3-phosphocholine; SpL: sphingolipid

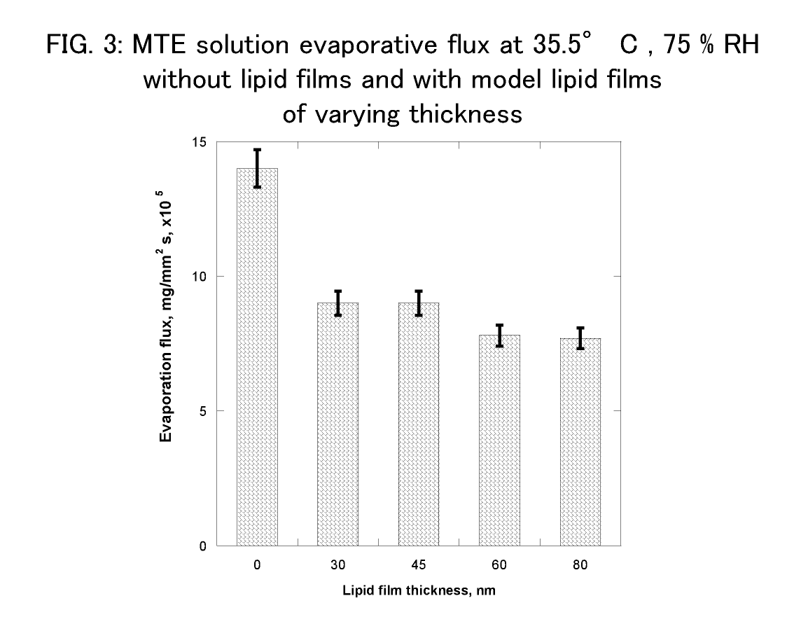

FIG. 3 depicts MTE solution evaporative flux at 35.5° C., 75% RH without lipid films and with model lipid films of varying thickness.

### Example 2

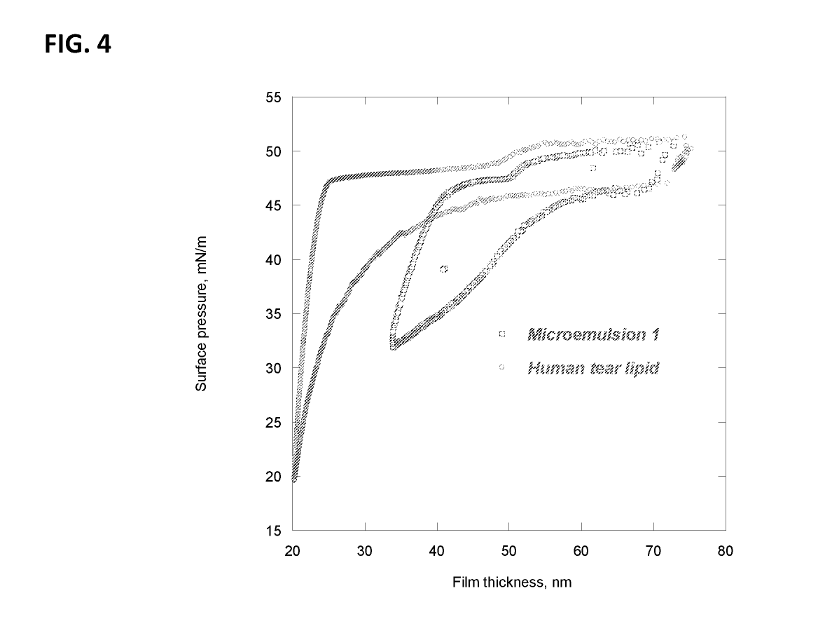

Microemulsion 1 contains only lipids and electrolytes naturally occurring in human tears. Microemulsion 1 has the interfacial pressure dynamics closely mimicking that of human tear lipids. The data are shown in FIG. 4. FIG. 4 depicts surface pressure (mN/m) as a function of lipid-film thickness for 1) human tear lipids; and 2) microemulsion #1.

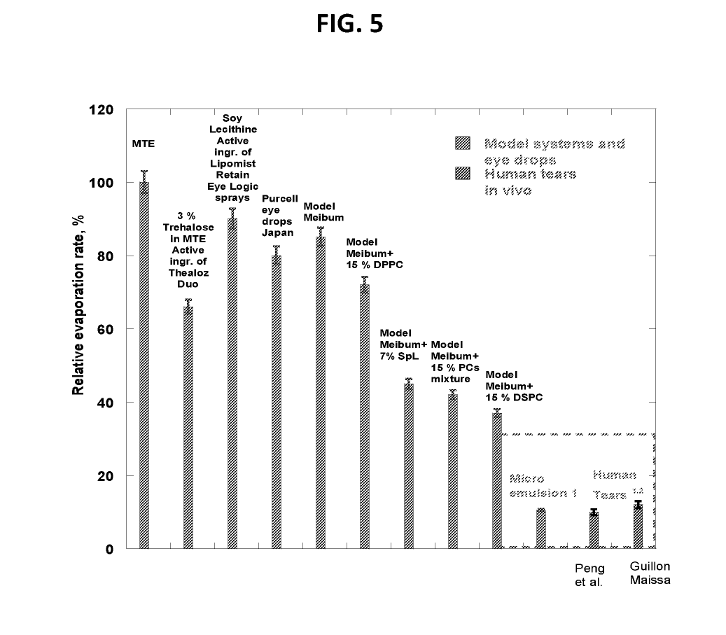

FIG. 5 provides a comparison of relative evaporation rates of model systems, microemulsion #1, commercial eye drops, and human tears in vivo. Microemulsion #1 reduces the evaporative flux to the level close to human tears in vivo. Evaporation rates of human tears in vivo were taken from: 1) Peng et al. (2014) Industrial & Engineering Chemistry Research 53:18130; and 2) Guillon and Maissa (2010) Contact Lens & Anterior Eye 33:171.

### Example 3

This example provides contralateral (Control solutions in one eye and Test solutions in the fellow eye) case studies in which in vivo performance of formulations of the present disclosure was observed, measured, and analyzed. No corneal staining or conjunctival redness was observed after application of a formulation of the present disclosure.

### Case#1

Case #1 was an asymptomatic female. The test solution was bicontinuous microemulsion #1. The controls were retain and saline solutions.

When compared to both Control solutions, the Test solution provided a long-lasting stability of the tear film up to 18 hrs with corresponding greater tear-lipid thickness as measured by interferometry, while the Control eye had decreased tear-lipid thickness. The masked subject reported better ocular comfort in the eye that was exposed to the Test solution compared to Baseline (no instilled solutions) and both Control solutions. Visual acuity was improved from 20/25 to 20/20 in test eyes in comparison with no changes with Saline control solution and decline to 20/30 in Retain control solution. No corneal staining or conjunctival redness was observed after application of a formulation of the present disclosure to the eye.

### Case #2

Case #2 was a symptomatic female. The test solution was microemulsion #2. The controls were Retain and saline solutions.

Compared to both Control solutions, the masked subject reported much better comfort in the eye that was exposed to the Test solution, and the effect lasted 6 hours. The subject also reported better comfort compared with Baseline and the effect lasted for 6 hrs. For the eye exposed to the Test solution, the stability of the tear film improved from 2 s at baseline to 5 s (30 min and 4 hrs after instilling Test solution), whereas the Control had deteriorating tear-film stability during the same time period for both Control solutions. The improved tear-film stability corresponded to improved functional vision. For example, low contrast visual acuity (VA) was improved from 20/60 at baseline to 20/40 (30 min, 2hr and 4hrs after instillation). High contrast VA was not changed before and after exposure of the Test solution, whereas it decreased with Retain solution Immediately upon instillation, the masked subject reported long-lasting blurriness with Retain solution compared with transient blurred vision, which dissipated within 30 sec, with the Test solution. No discomfort was reported upon instillation of Test or Control solutions. Subject reported less vision fluctuation in the Test eye. No corneal staining or conjunctival redness was observed after application of a formulation of the present disclosure to the eye.

### Case #3

Case #3 was an asymptomatic female. The test solution was microemulsion #2. The controls were Retain and saline solutions.

Clinical results were very similar to those found in Case #2. However, the impact was not as great in this asymptomatic subject compared with the symptomatic subject. One notable finding is that even given the smaller magnitudes of improvement in tear-film stability from Baseline or compared with the Control solutions, the improvement was long lasting (>12 hrs). Therefore, this asymptomatic subject could discern a difference in comfort between Test and Control eyes for at least 24 hours. No corneal staining or conjunctival redness was observed after application of a formulation of the present disclosure to the eye.

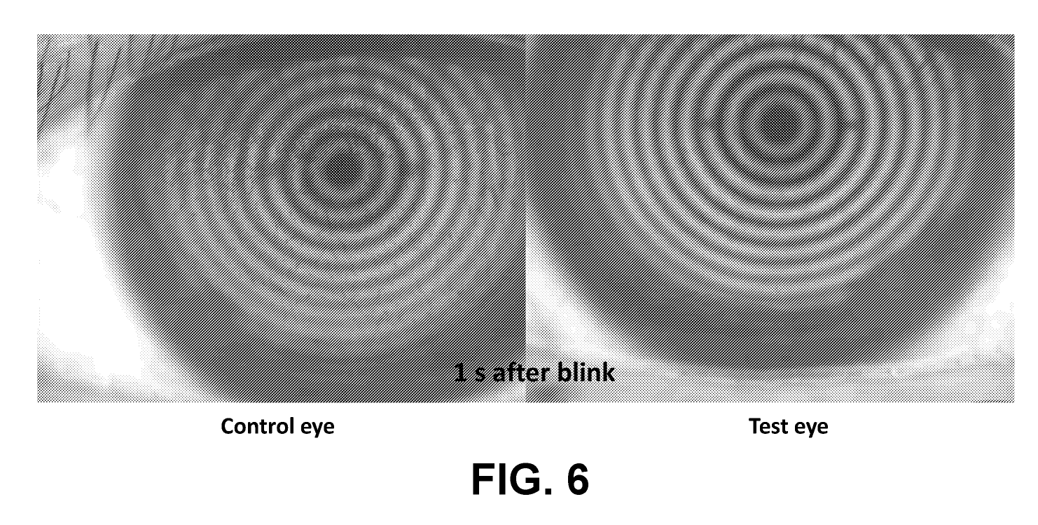

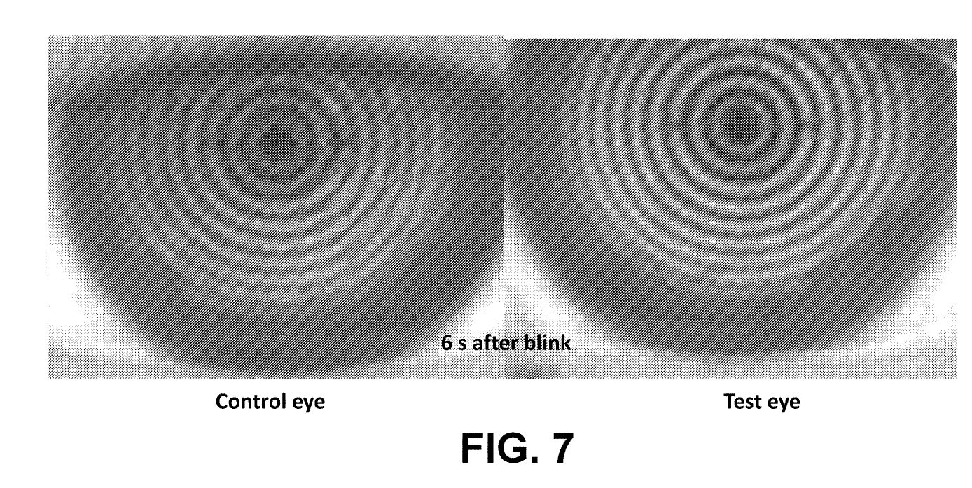

In summary, the test formulations stabilized tear film with long-lasting effect while not compromising vision quality. The test formulations can greatly reduce ocular discomfort due to dryness and tear-film instability. In addition, these microemulsion drops can improve ocular imaging quality (anterior and posterior segments) by stabilizing the air-tear film interface of the optical path. This is because the tear-lipid-film quality was substantially improved after Test formulation drop instillation, as demonstrated in FIG. 6 and FIG. 7. The lipid film of the Test eye became more uniform in thickness and more evenly distributed over the entire cornea. These improvements, far more pronounced in the eyes of the symptomatic subject, are the most likely reasons of enhanced visual acuity as well as improved ocular-imaging quality. One example of ocular imaging of the anterior segment that can be benefited from a stabilized air-tear film interface is corneal topography. A stable air-tear film interface can lead to a better approximation of the corneal power, leading to improved accuracy of calculated crystalline lens powers prior to a cataract surgery.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A bicontinuous microemulsion comprising:
a) an aqueous phase comprising one or more of:
i) sodium chloride;
ii) sea salt;
iii) ascorbic acid; and
iv) trehalose; and
b) a lipid phase comprising one or more of:
i) sphingomyelin;
ii) phosphatidylcholine;
iii) fish oil;
iv) palm oil;
v) olive oil,
vi) coconut oil;
vii) grapeseed oil;
viii) bees wax;
xi) ghee; and
x) lanolin,
wherein the osmolarity of the microemulsion is from 270 mOsm to 320 mOsm, and wherein the microemulsion has a pH of from 7.2 to 7.4.

2. The bicontinuous microemulsion of claim 1, wherein the ratio of aqueous phase to lipid phase is from about 10:1 to 1:10 (v/v).

3. The bicontinuous microemulsion of claim 1, wherein the ratio of aqueous phase to lipid phase is from about 5:1 to 1:5 (v/v).

4. The bicontinuous microemulsion of claim 1, wherein the ratio of aqueous phase to lipid phase is from about 3:1 to 1:3 (v/v).

5. The bicontinuous microemulsion of claim 1, wherein the ratio of aqueous phase to lipid phase is from about 2.5:1 to 3:1 (v/v).

6. The bicontinuous microemulsion of any one of claims 1-5, comprising lactoferrin.

7. The bicontinuous microemulsion of any one of claims 1-6, wherein the lipid phase comprises sphingomyelin and phosphatidylcholine.

8. The bicontinuous microemulsion of claim 7, wherein the ratio of sphingomyelin to phosphatidylcholine is from 4:1 to 1:1 (w/v).

9. The bicontinuous microemulsion of any one of claims 1-6, wherein the lipid phase comprises palm oil and olive oil.

10. The bicontinuous microemulsion of claim 9, wherein the ratio of palm oil to olive oil is from 3:1 to 1:3 (v/v).

11. The bicontinuous microemulsion of any one of claims 1-6, wherein the lipid phase comprises palm oil, olive oil, and fish oil.

12. The bicontinuous microemulsion of claim 11, wherein the ratio of palm oil and olive oil to fish oil is from 10:1 to 6:1 palm oil+olive oil:fish oil.

13. The bicontinuous microemulsion of any one of claims 1-7, comprising coconut oil.

14. The bicontinuous microemulsion of any one of claims 1-7, wherein the lipid phase comprises coconut oil or palm oil; olive oil; and fish oil.

15. The bicontinuous microemulsion of claim 14, wherein the ratio of palm oil/coconut oil and olive oil to fish oil is from 10:1 to 6:1 palm oil/coconut oil+olive oil:fish oil.

16. The bicontinuous microemulsion of any one of claims 1-7, wherein the lipid phase comprises nonpolar lipids and polar lipids in a ratio of from about 10:1 to 4:1 (v/v).

17. The bicontinuous microemulsion of any one of claims 1-7, wherein the lipid phase comprises grapeseed oil, coconut oil, beeswax, sphingomyelin, and phosphatidylcholine.

18. The bicontinuous microemulsion of any one of claims 1-7, wherein the lipid phase comprises palm oil, beeswax, lecithin, and phosphatidylcholine.

19. The bicontinuous microemulsion of any one of claims 1-7, wherein the lipid phase comprises ghee, lecithin, and phosphatidylcholine.

20. The bicontinuous microemulsion of any one of claims 1-7, wherein the lipid phase comprises ghee, lecithin, sphingomyelin, and phosphatidylcholine.

21. The bicontinuous microemulsion of any one of claims 1-7, wherein the lipid phase comprises grapeseed oil, beeswax, lecithin, and sphingomyelin.

22. The bicontinuous microemulsion of any one of claims 1-7, wherein the lipid phase comprises grapeseed oil, coconut oil, beeswax, lecithin, and sphingomyelin.

23. The bicontinuous microemulsion of any one of claims 1-7, wherein the lipid phase comprises ghee, lecithin, and sphingomyelin.

24. The bicontinuous microemulsion of any one of claims 1-7, wherein the lipid phase comprises coconut oil and grapeseed oil.

25. The bicontinuous microemulsion of claim 1, wherein the lecithin is egg yolk lecithin.

26. The bicontinuous microemulsion of any one of claims 1-25, wherein the bicontinuous emulsion is sterile.

27. A formulation comprising the bicontinuous microemulsion of any one of claims 1-26, wherein the formulation is a liquid, a gel, a semi-solid, or an ointment.

28. A container comprising the bicontinuous microemulsion of any one of claims 1-26 or the formulation of claim 27.

29. The container of claim 28, wherein the container comprises an eyedropper.

30. A method of reducing evaporation of an aqueous tear film from the surface of an eye, the method comprising applying the bicontinuous microemulsion of any one of claims 1-26 or the formulation of claim 27 to the surface of the eye, thereby reducing evaporation of the aqueous tear film from the surface of the eye.

31. A method of treating dry eye in an eye of an individual, the method comprising applying the bicontinuous microemulsion of any one of claims 1-26 or the formulation of claim 27 to the surface of the eye of the individual.
